(19) United States
(12) Patent Application Publication
(10) Pub. No.: US 2011/0278175 A1
(43) Pub. Date: Nov. 17, 2011

(54) Aniline Derivatives, Polymers, and Uses Thereof

(76) Inventors: Michael James WHITCOMBE, Norwich (GB); Sergey Anatolievich Piletsky, Cranfield (GB); Dhana Lakshmi, Cranfield (GB); Petya Krasimirova Ivanova-Mitseva, Cranfield (GB)

(21) Appl. No.: 13/106,095

(22) Filed: May 12, 2011

(30) Foreign Application Priority Data
May 12, 2010 (GB) 10079051.1

(51) Int. Cl. C08G 75/00; C25B 3/02; C08F 283/00; C07C 333/20; B82Y 30/00

(52) U.S. Cl. 205/414; 528/390; 525/535; 558/236; 977/773; 977/762

(57) ABSTRACT

A monomer having an aniline moiety linked through nitrogen to a dithiocarbamate moiety, e.g. N—(N¹,N¹-diethyldithiocarbamoylethylamidoethyl)-aniline:

Ph-NH—CH₂—CH₂—NH—CO—CH₂—CH₂—S—C(═S)—NEt₂

can be oxidatively polymerised to produce a polyaniline bearing dithiocarbamate moieties. This can be used as an iniferter, to graft addition polymers onto it.

### BACKGROUND OF THE INVENTION

1. Field of the Invention

The invention relates to aniline derivatives, to polymers and copolymers thereof, and to uses of such polymers and copolymers, which are generally conjugated.

2. Description of the Prior Art

The formation of a conjugated polymer bearing double bonds, by polymerisation or electropolymerisation of a conjugated polymer precursor bearing the said double bond was disclosed in WO2009/118525, in particular the monomer N-phenylethylenediamine methacrylamide (NPEDMA) (also known as N-(methacrylamidoethyl)-aniline (NMAEA)) has been shown to be capable of chemical or electrochemical polymerisation to form polyaniline materials in various formats capable of utilisation in other reactions, such as participation in free radical-initiated addition polymerisation after formation of the polyaniline material while preserving or even enhancing some of the properties (such as electrical conductivity) of the polyaniline [1]. It has further been shown that the same material can be utilised as the basis of the construction of an electrochemical sensor for catechol by grafting of a layer of a catalytically-active MIP over the polyaniline layer [2]. The electrochemical sensor for catechol (and dopamine) constructed by this method was shown to be superior to electrodes prepared by direct immobilisation of MIP particles of essentially the same composition to the surface of a suitable electrode. This phenomenon was explained by the conjugated polymer acting as a “molecular wire” due to the more intimate contact between the catalytically-active imprint sites and the conjugated polymer layer than achievable by immobilisation of electrically-insulating polymer on an electrode. Grafting of the MIP layer however could not be performed directly, but required an additional activation step, in which the double-bond bearing polyaniline layer was irradiated in the presence of an iniferter compound, in this case N,N-diethyldithiocarbamic acid benzyl ester, resulting in the partial conversion of the pendant double bonds present on the polyaniline structure into groups capable of initiating further polymerisation. However it would be desirable to avoid the use of an additional activation step which could be achieved if the conjugated polymer precursor was already derivatised with dithiocarbamate ester groups rather, than with double bonds. Such a material would allow the formation of conjugated polyanilines, derivatised with dithiocarbamate ester groups capable of directly activated grafting of addition polymers or of any other reactions associated with dithiocarbamate ester derivatised polymers.

The synthesis of double bond-derivatised addition monomers bearing dithiocarbamate esters and their polymerisation to macroiniferters has previously been reported [3-7] as has the post-derivatisation of polymeric materials, such as polymers and copolymers of 4-vinyl chloromethylbenzene [8,9]. Reports of the formation of conjugated polymers bearing dithiocarbamate groups have been limited. U.S. Pat. No. 7,649,048 discloses polymeric macroiniferters based on poly(vinylene phenylene) with pendant dithiocarbamate ester side groups. The macroiniferter disclosed in U.S. Pat. No. 7,649,048 is not however formed by the polymerisation of a monomeric precursor, but rather from the partial elimination of dithiocarbamate ester groups from a precursor polymer. In the case of this material, elimination of dithiocarbamate groups is responsible for introduction of the vinylene group, which is responsible for formation of the conjugated polymer backbone [as disclosed in U.S. Pat. No. 7,446,164], therefore the partial conversion, in order to leave dithiocarbamate ester groups capable of grafting, necessarily results in a break in the conjugated structure of the polymeric backbone.

### REFERENCES

1. Lakshmi, D., Whitcombe, M. J., Davis, F., Chianella, I., Piletska, E. V., Guerreiro, A. R., Subrahmanyam, S., Brito, P. S., Fowler, S. A., & Piletsky, S. A., 2009, Chimeric Polymers Formed from a Monomer Capable of Free-Radical, Oxidative and Electrochemical Polymerisation. Chem. Commun., 2759-2761.
2. Lakshmi, D., Bossi, A., Whitcombe, M. J., Chianella, I., Fowler, S. A., Subrahmanyam, S., Piletska, E. V. & Piletsky, S. A. 2009, Electrochemical Sensor for Catechol and Dopamine Based on a Catalytic Molecularly Imprinted Polymer-Conducting Polymer Hybrid Recognition Element. Anal. Chem., 81 (9), 3576-3584.
3. Otsu, T., Yamashita, K. & Tsuda, K., 1986, Synthesis, reactivity, and role of 4-vinylbenzyl N,N-diethyldithiocarbamate as a monomer-iniferter in radical polymerization. Macromolecules, 19, 287-290.
4. Qin, S. H. & Qiu, K. Y., 2001, A new polymerizable photoiniferter for preparing poly(methyl methacrylate) macromonomer. Eur. Polym. J. 37, 711-717
5. Luo, N., Hutchison, J. B., Anseth, K. S. & Bowman, C. N. 2002, Synthesis of a novel methacrylic monomer iniferter and its application in surface photografting on crosslinked polymer substrates. J. Polym. Sci. Part A, 40, 1885-1891.
6. Garcia-Con, L. M., Whitcombe, M. J., Piletska, E. V., Piletsky, S. A., 2010 A sulfur-sulfur cross-linked polymer synthesized from a polymerizable dithiocarbamate as a source of dormant radicals. Angew. Chem. Int. Ed. 49, 4075-4078.
7. Ivanova-Mitseva, P. K., et al., Macromolecules, 44, 1856-1865.
8. Sellergren, B., Rückert, B. & Hall, A. J., 2002, Layer-by-layer grafting of molecularly imprinted polymers via iniferter modified supports, Adv. Mater., 14, 1204-1208.
9. Pérez-Moral, N. & Mayes, A. G., 2007, Molecularly Imprinted Multi-Layer Core-Shell Nanoparticles—A Surface Grafting Approach. Macromol. Rapid Commun., 28, 2170-2175.
10. Bossi, A., Whitcombe, M. J., Uludag, Y., Fowler, S., Chianella, I., Subrahmanyam, S. Sanchez, I. & Piletsky, S. A. 2010, Synthesis of controlled polymeric cross-linked coatings via iniferter polymerisation in the presence of tetraethyl thiuram disulphide chain terminator. Biosens. Bioelectron. 25, 2149-2155.

Patent References

| Pat. | Issued | Title |
|---|---|---|
| WO2009118525 | 01-10-2009 | Conjugated interpenetrated |
| | | polymer networks |
| U.S. Pat. No. 7,649,048 | 19-01-2010 | Conjugated polymers provided |
| | | with at least one molecular |
| | | imprinted polymer and a method |
| | | for their preparation via |
| | | conjugated macro-iniferters |
| U.S. Pat. No. 7,446,164 | 04-11-2008 | Method of preparing derivatives |
| | | of polyarylene vinylene and |
| | | method of preparing an |
| | | electronic device including same |
| U.S. Pat. No. 2,786,866 | 26-03-1957 | Esters of dithiocarbamic acids |
| | | and a method for their |
| | | preparation |
| U.S. Pat. No. 4,381,401 | 26-04-1983 | Aminoethylation process |

### SUMMARY OF THE INVENTION

In a first aspect the invention provides a monomer having an aniline moiety linked through the aniline nitrogen to a dithiocarbamate moiety. The linkage is generally via a spacer unit.

The linkage may be to a sulfur atom of the dithiocarbamate moiety so that the monomer is of formula (A):

Ar—NH-[SPACER]-S—C(═S)—NR¹R² (A)

The spacer generally comprises a chain of 2-7 carbon atoms, one or more of which may be replaced by a heteroatom, particularly O or N.

Some of the atoms in the chain may be functionalised or bear substituents (e.g. OH, C₁₋₄ alkyl). Particularly preferred spacers comprise ester or amide linkages. Examples include:

—CH₂—CH₂—X—CO—CH₂—CH₂—

(in either orientation), where X is O or NH;

—(CH₂)ₙ—

where n is 2-7;

—(CH₂)ₐ—O—(CH₂)_{b}—

where a and b are integers of 1-5 and a+b is 2-6; and variants in which one or more methylene groups are substituted.

Alternatively the linkage may be to the nitrogen atom of the dithiocarbamate moiety, so that the monomer is of formula (B):

Ar—NH-[SPACER]-NR³—C(═S)—S—R⁴ (B)

where the spacer may be selected from the same possibilities as in formula (A), or may be —CH₂— or a substituted methylene.

In formulas (A) and (B), Ar may be an unsubstituted phenyl group or may have up to 4 substituents which are the same or different.

The groups R¹, R² and R³ may each be of the form —CH₂R⁵ where R⁵ is H or an optionally substituted straight or branched alkyl group, preferably of 1-6 carbon atoms, more preferably of 1-3 carbon atoms. R¹ and R² may be the same or different, or may be linked to form a cyclic structure. The alkyl or alkylene groups may be interrupted by heteroatoms, e.g. oxygen.

R⁴ may be a straight chain or branched alkyl group, optionally substituted, e.g. optionally substituted benzyl or diphenylmethyl. The alkyl component may have 1-6 carbon atoms, preferably 1-3 carbon atoms (disregarding any substituents).

Alternatively a monomer is in the form of a salt, protonated on the aniline nitrogen, with an anion that may be derived from a mineral acid or an organic (e.g. sulfonic) acid.

In a second aspect the invention provides a polymer or copolymer produced by polymerising one or more monomers comprising a monomer according to the first aspect, polymerised through the aniline moiety. Preferred comonomers are also anilines. A preferred polymeric material is a conjugated polyaniline material wherein at least some aniline units bear dithiocarbamate groups.

In a third aspect the invention provides a method of producing a polymer or copolymer of the second aspect by polymerising monomer(s) comprising a monomer of the first aspect, e.g. electrochemically or by chemical oxidation.

Thus the invention includes various compositions of matter comprising monomers based on aniline bearing dithiocarbamate ester groups attached to the nitrogen atom through a spacer group, and their salts comprising the said aniline derivative protonated on nitrogen and their polymers and copolymers, polymerised through the aniline functionality, which comprise a conjugated polyaniline material bearing a high density of dithiocarbamate ester groups. The monomers of the current invention may be polymerised either electrochemically or through oxidation of the aniline function with suitable chemical oxidising agents such as ammonium persulphate or any other suitable oxidising agent known to those skilled in the art to form the substituted polyaniline (PANI) materials of the present invention in which some or all of the monomer residues are substituted with the dithiocarbamate group. Furthermore the present invention also includes copolymers of the monomers of the current invention with one or more additional aniline-based monomer, which can be unsubstituted, substituted with one or more functional groups on the ring or with one substituent on nitrogen. Polymerisation of the aniline derivatives, either by electrochemical or chemical oxidation can be carried out on the protonated salts of the monomers of the present invention, either with or without addition of further acid, or on the unprotonated aniline derivative in the presence of added acid, such as HCl or any other proton source known to those skilled in the art. Polymerisation of the aniline functionality can be performed in purely aqueous or mixed aqueous-organic mixtures or purely organic media according to those methods known to those skilled in the art, moreover the solvent or solvent mixture may include an additional proton source and optionally include other components such as electrolytes or surfactants. Polymers or copolymers of the monomers or salts of the monomers of the present invention can be prepared as thick or thin films, powder, particles, microparticles, nanoparticles, microcapsules, microtubes, microrods, nanotubes or nanorods or as soluble polymers or in any other form or geometry known to those skilled in the art. The polymers of the present invention may also be optionally doped through protonation, treatment with iodine or using any other techniques known to specialists in the chemistry of conjugated polymers.

The dithiocarbamate ester functionality of the monomers of the present invention may comprise a dithiocarbamate ester group bearing two substituents on nitrogen, linked through at least one —CH₂— linkage such that the substituents are alkyl, substituted or branched alkyl chains or are linked such that the two substituents form a cyclic structure, either comprising a series of linked methylene units or substituted methylene units, including examples where one or more methylene groups are substituted by a heteroatom such as oxygen. Examples of the substituents covered by the present invention include, but are not limited to: dimethyl; diethyl; di(hydroxyethyl); methyl ethyl; methyl butyl; dibutyl; and cycles comprising —(CH₂)₄—; —(CH₂)₅—; —(CH₂)₂—O—(CH₂)₂— and others known to those skilled in the art, the aniline functionality being coupled to the dithiocarbamate ester group through the S atom. Furthermore the monomers of the present invention also include materials where the aniline derivative is present as one of the substituents on the dithiocarbamate ester nitrogen, as well as monomers where the aniline group is present as a substituent on the S atom of the dithiocarbamate ester group. In the case of monomers where the aniline substituent is attached to the dithiocarbamate ester nitrogen atom, the ester substituent on the S atom of the dithiocarbamate group may be an alkyl substituent such as methyl, ethyl, butyl or benzyl, or any other such substituent know to those skilled in the art.

The dithiocarbamate esters groups of the monomers and polymers of the present invention are therefore examples of compounds well-known to be capable of formation of a pair of free radicals on irradiation with ultra-violet light which have very different reactivities, such that a reactive carbon-centred radical and a low reactivity (sometimes know as dormant) dithiocarbamate radical are formed on photochemical cleavage of the C—S bond. Furthermore under certain conditions the dithiocarbamate ester groups are reformed through radical recombination reactions, such that the photochemical cleavage is a reversible process to reform the initial dithiocarbamate group or a new dithiocarbamate group, which is also capable of photochemical cleavage to form a new pair of radicals. Furthermore the different reactivities of the radical pair can be exploited in reactions known to be carried out by radicals of this type known to those skilled in the art, in particular dithiocarbamate esters of the type described in this invention are well know to act as initiators of polymerisation of unsaturated monomers such as acrylate esters, methacrylate esters, acrylamides, methacrylamides, vinyl aromatics, vinyl esters, vinyl amides and other unsaturated monomers capable of free radical polymerisation known to those skilled in the art, such that propagation of polymerisation will occur under conditions of radical formation such as UV irradiation, whereas dithiocarbamate esters will be reformed by radical recombination reactions on removal of the source of UV, the newly formed dithiocarbamate esters also being capable of re-initiating polymerisation with the same or different monomer or monomers upon reapplication of the source of UV light. This property of dithiocarbamate esters imparts “living” or more strictly “pseudo-living” character to photochemical polymerisations initiated by irradiation of dithiocarbamate esters, which are know as photochemical “iniferters”, the latter description relating to there role as initiator, chain transfer agent and terminators. An important property of living polymerisations, including those initiated with photochemical iniferters, is their ability to be “switched on” and “switched off” by the application of the initiating light source, such that polymerisation of unsaturated monomers may be performed in a controlled manner, including re-initiation in the presence of a different monomer mixture such that block copolymers may be formed. Furthermore other reactions of free radicals derived from dithiocarbamate ester groups, such as hydrogen abstraction and cross-coupling of carbon-centred and/or dormant radical pairs can be exploited to perform other functions such as immobilisation of biological molecules and/or cross-linking of polymers bearing the dithiocarbamate ester groups, either to form permanent or reversible cross-links depending on the structure of the monomer.

Activation of the dithiocarbamate ester-based iniferter groups of the materials of the present invention to allow grafting of addition polymers, copolymers or block copolymers, can either be carried out before polymerisation of the aniline part to form a telechelic polymer bearing the aniline group at one of the chain termini of the addition polymer, copolymer or block copolymer, which can be further polymerised to a polymer-grafted polyaniline. Alternatively activation of the dithiocarbamate ester group in the presence of unsaturated monomers can be performed after formation of polyaniline, through electrochemical or chemical oxidation of the aniline functionality, in this case resulting in grafting of one or more layers of addition polymers or copolymers, including linear or cross-linked polymer grafts or combinations of linear and cross-linked layers. In the case of grafting of cross-linked polymers, they may be optionally formed in the presence of a mixture of monomers and in the presence of one or more molecular, macromolecular, protein, peptide, ionic, crystal, viral, bacterial, yeast or other template known to those skilled in the art, to form a molecularly imprinted polymer (MIP) layer as one or more of the grafted layers.

Polymerisation of the aniline functionality of the monomers of the present invention can therefore be performed on a number of substrates, including, but not limited to: indium-tin oxide-coated glass, gold, platinum, carbon or other electrodes, metallic, polymeric, glassy or ceramic surfaces or as suspensions of particles, microparticles, nanoparticles or as microcapsules, or as micro- or nano-rods or tubes, either as free suspensions or anchored to a surface, or as soluble polymers depending on the solvent and reaction conditions or the use of template structures, such as nanoporous membranes, or as soluble polymers or by any other such method known to those skilled in the art. In particular the monomers of the present invention will be useful for coating electrodes and transducer chips, including optical, acoustic or electrochemical sensors with polyaniline coatings which can be further derivatised or functionalised by photochemical activation of the dithiocarbamate ester groups to graft addition polymers based on vinyl monomers, including the grafting of molecularly imprinted polymers or to immobilise biomolecules, natural or artificial receptor molecules, antibodies, enzymes, nanoparticles, proteins, aptamers, cells or other species known to those skilled in the art to be useful in the construction of sensor for the detection and quantification of chemical or biological species, using methods known to those skilled in the art. In addition polymerisation of the monomers of this invention, particularly by polymerisation using chemical oxidising agents, such as persulphate, can be used to coat a range of articles, such as micro-titre plates, cuvettes, membranes, filter elements, fibres, tubes, catheters, injection-moulded, cast, woven and sintered articles with a layer of dithiocarbamate-functionalised polyaniline as a means to further coat the material with a layer of grafted addition polymer, including but not limited to, grafted layers of molecularly imprinted polymer, to impart additional surface properties to the articles. Furthermore polymerisation of functional graft polymers and copolymers, to the dithiocarbamate ester-functionalised polyaniline of the present invention, either in solid form or as a solution, can be used to form soluble polyanilines with modified properties, for example to form self-doped soluble polyaniline molecules or to form soluble polyaniline bearing additional functionality for the attachment of biomolecules for use in biosensor or for coating applications or any other application known to those skilled in the art. As mentioned above, U.S. Pat. No. 7,649,048 discloses macroiniferters which, because of their manner of synthesis, have breaks in the conjugation.

This is not the case in the materials described in the present invention since the conjugated polyaniline backbone is formed in a process completely independent of the introduction of the dithiocarbamate ester groups. Furthermore the macroiniferters disclosed in U.S. Pat. No. 7,649,048 cannot carry a high density of dithiocarbamate ester groups, also for the reason that the conjugated structure is only created by elimination of dithiocarbamate residues. This is contrary to the materials disclosed in the present invention, for which each of monomer residues is potentially capable of carrying one dithiocarbamate ester residue, therefore there is no trade-off between backbone conjugation and the number of pendant dithiocarbamate ester groups.

### BRIEF DESCRIPTION OF THE DRAWINGS

FIG. 1. Structures of some of the monomers disclosed in the present invention, I-III and their corresponding protonated forms IV-VI.

FIG. 2. Cyclic voltammogram obtained during the electropolymerisation of NDDEAEA (0.1M in 0.75M HCl, 25% AcN) to form a film of poly(NDDEAEA) on the working electrode of a gold screen-printed electrode at a scan rate of 100 mV s⁻¹.

FIG. 3. Schematic representation of the photografting of layers of poly(MAA) and poly(styrene) to an electropolymerised poly(NDDEAEA) on a gold electrode, including the layer-by-layer grafting of poly(styrene) over poly(MAA).

FIG. 4. 3-D bar charts showing: a) The effect of monomer concentration and HCl concentration on the optical density of chemically polymerised films of poly(NDDEAEA) at constant (0.0183 M) ammonium persulphate concentration (measured at pH 1) and b) the effect of monomer concentration and ammonium persulphate concentration on the optical density of chemically polymerised films of poly(NDDEAEA) at constant (0.225 M) HCl concentration (measured at pH 1).

FIG. 5. ¹H-NMR spectrum of poly(NDDEAEA) in d₆-DMSO, recorded at 50° C. (JEOL EX-400), and the structure of the corresponding monomer. The presence of the diethyldithiocarbamate groups is shown by the presence of the N-ethyl groups at around 1.1 ppm (methyl) and 3.8 ppm (N-substituted methylene). The connectivity of the remaining methylene groups, as indicated on the spectrum, was determined from the corresponding COSY spectrum.

FIG. 6. UV spectrum of soluble polyNDDEAEA in methanol, showing absorption peaks at λₘₐₓ=247 and 275 nm which are attributed to the —S—C═S and S═C—N groups of diethyl dithiocarbamates respectively. Also the spectrum shows three characteristic absorption bands due to polyaniline at around 320, 400 and 735 nm. The characteristic peaks of PANI appear at about 320 nm due to the π-π* transition of the benzenoid ring and at about 400-430 nm and 730-825 nm due to polaron-π* and π-polaron band transitions respectively, showing that the PAN™ is present in the doped state.

### DETAILED DESCRIPTION OF THE INVENTION

This invention relates to various compositions of matter comprising monomers based on aniline bearing dithiocarbamate ester groups attached to the nitrogen atom through a spacer group, and their salts comprising the said aniline derivative protonated on nitrogen and their polymers and copolymers, polymerised through the aniline functionality, which comprise a conjugated polyaniline material bearing a high density of dithiocarbamate ester groups, as well as processes involving applications of the said materials.

### First Embodiment

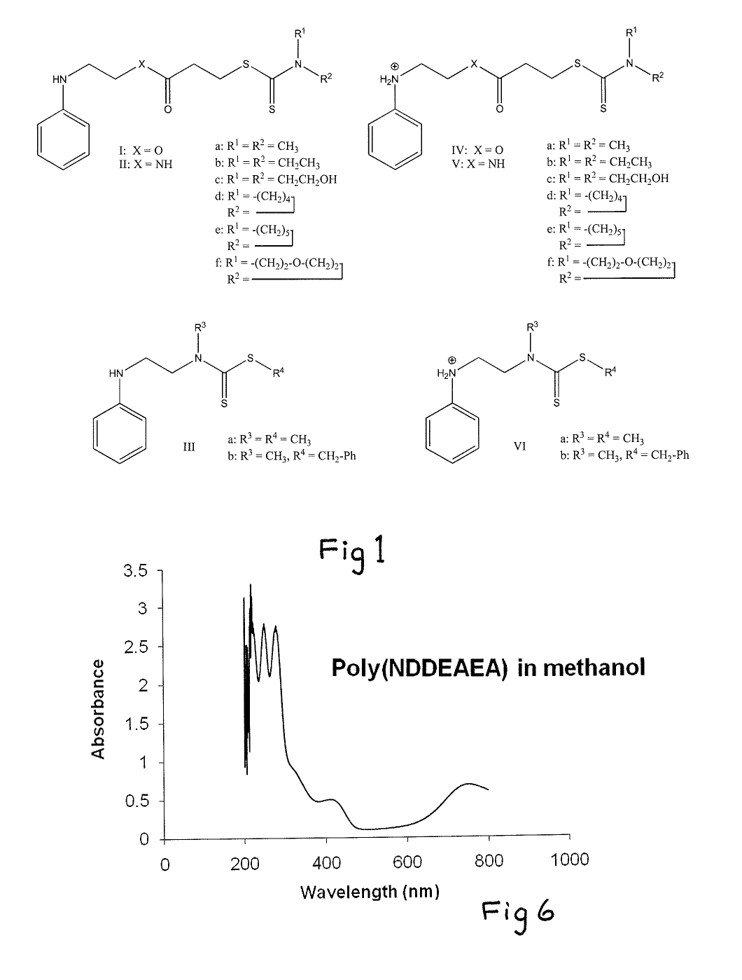

The monomers of the present invention may comprise an aniline group, substituted with a spacer on the aniline nitrogen atom, connected to the S atom of a dithiocarbamate ester group, bearing two alkyl substituents on nitrogen. The spacer may consist of between 2 to 6 atoms, preferably consisting of 6 atoms, including an amide or ester group, preferably an amide group in the spacer. Preferably the monomer is prepared by coupling of a dithiocarbamate ester of propanoic acid, prepared by reaction of acrylic acid, CS₂ and a secondary amine in the presence of base (prepared as disclosed in U.S. Pat. No. 2,786,866) with N-phenylethylene diamine or with N-(2-hydroxyethyl)aniline to form the amide-linked or ester linked monomers respectively (II and I in FIG. 1). The nitrogen atom of the dithiocarbamate ester group is doubly substituted, the substitution deriving from the above mentioned secondary amine used in the preparation of the dithiocarbamate ester of propanoic acid. The two substituents on nitrogen, are linked through at least one —CH₂— linkage such that the substituents are alkyl, substituted or branched alkyl chains or are linked such that the two substituents form a cyclic structure, either comprising a series of linked methylene units or substituted methylene units, including examples where one or more methylene groups are substituted by a heteroatom such as oxygen. Examples of the substituents covered by the present invention include, but are not limited to: dimethyl; diethyl; di(hydroxyethyl); methyl ethyl; methyl butyl; dibutyl; and cycles comprising —(CH₂)₄—; —(CH₂)₅—; —(CH₂)₂—O—(CH₂)₂— and others known to those skilled in the art. Some examples of the structures of the monomers of the current invention are represented by the structures Ia-f and IIa-f in FIG. 1, although practitioners in the art will realise that the monomers of the current invention are not limited to these few examples, but that they only represent a small subset of the materials consistent with this embodiment, furthermore the monomers of this embodiment may further be prepared as salts, by protonation of the nitrogen atom to form the corresponding structures represented by the structures IVa-f and Va-f in FIG. 1, by reaction with a mineral acid or a sulphonic acid or other such proton source known to those skilled in the art, preferably by reaction with HCl.

### Second Embodiment

The monomers of the present invention may also comprise dithiocarbamate esters in which the N-substituted aniline group is alternatively present as one of the substituents bound to the nitrogen atom of the dithiocarbamate ester group, in this case the second substituent bound to the dithiocarbamate nitrogen atom is an alkyl substituent, a branched alkyl substituent or a substituted alkyl substituent, examples being methyl, ethyl, hydroxyethyl, propyl or butyl or any other such group as known to practitioners in the art, preferably the alkyl substituent is a methyl group. In this embodiment the group bound to the S atom of the dithiocarbamate ester group is an alkyl substituent or benzyl group or any other suitable group known to those skilled in the art. Some examples of monomers covered by this embodiment of the invention are represented by the structures IIIa-b in FIG. 1, although practitioners in the art will realise that these structures are only representative and that the monomers of this embodiment are not limited to this small subset of materials. In addition this embodiment also encompasses the corresponding monomer structures where the nitrogen atom of the aniline group is protonated to form a salt of the monomer, as represented by, but not limited to the structures VIa-b, FIG. 1, by reaction with a mineral acid or a sulphonic acid or other such proton source known to those skilled in the art, preferably by reaction with HCl. In one preferred embodiment, the monomers IIIa-b can be prepared by the reaction of N-methyl-N′-phenyl-1,2-ethylenediamine with carbon disulphide and methyl iodide (in the case of IIIa) or benzyl bromide (in the case of IIIb), or by any other such method known to those skilled in the art. N-methyl-N′-phenyl-1,2-ethylenediamine in turn can be prepared by a number of synthetic routes, including by reaction of aniline hydrochloride with 3-methyl-2-oxazolidinone in 2-(2-methoxyethoxy)ethanol, as disclosed in U.S. Pat. No. 4,381,401 or by any other such method known to those skilled in the art.

### Third Embodiment

In a further embodiment of the materials of the present invention, polymeric films of dithiocarbamate-functionalised polyaniline can be formed by electropolymerisation of the aniline monomers of the present invention, optionally in the presence of one or more additional aniline-based monomer, by for example, cyclic voltammetry, over gold, platinum or carbon or other suitable electrode material known to those skilled in the art, in aqueous or mixed aqueous organic solution or in purely organic solution, as known to those skilled in the art, preferably in mixed organic aqueous solution, preferably in aqueous acetonitrile. The electropolymerisation can be performed on solutions of the aniline monomer or of the protonated form of the aniline monomer, the solution containing an additional proton source (optionally in the case of the protonated form of the monomer) and an electrolyte salt, preferably under conditions which result in the deposition of a layer of the substituted polyaniline material upon the electrode surface.

### Fourth Embodiment

In a further embodiment of the present invention, dithiocarbamate ester-based polyaniline may be prepared by treatment of the aniline-based monomers of the present invention, or their protonated salts, optionally in the presence of one or more additional aniline-based monomers, with one or more chemical oxidants such as ammonium persulphate or other suitable oxidants known to those skilled in the art, in aqueous solution or mixed aqueous-organic solution or in purely organic solution, in the presence of an additional proton source and optionally in the presence of surfactant or in the presence of an article to be coated with the polyaniline material, or in the presence of an interface or any other device or method know to those skilled in the art to influence the shape or morphology of the polyaniline materials. The dithiocarbamate-based polyaniline materials may therefore be formed as thick or thin films, powder, particles, microparticles, nanoparticles, microcapsules, microtubes, microrods, nanotubes or nanorods or as soluble polymers or in any other form or geometry known to those skilled in the art.

### Fifth Embodiment

In a further embodiment of the present invention, the dithiocarbamate ester-derivatised polyaniline layers, articles or solutions are capable of being used as macroiniferter to graft addition polymers either as homopolymers or copolymers of linear or cross-linked polymer under conditions favouring initiation of polymerisation by dithiocarbamate ester, i.e. UV-irradiation in substantially oxygen-free solutions of unsaturated monomer, optionally including addition of N,N,N′,N′-tetraethyl dithiuram disulphide or other such additional source of dormant radicals, to provide further control in the case of surface-confined grafting [10]. Suitable unsaturated monomers are selected from, but not limited to, representatives from the group consisting of vinyl, allyl, styrene, acrylic, methacrylic or acetylene derivatives, with non-exclusive examples of divinylbenzene, divinylnaphthalene, vinylpyridine, hydroxyalkylene methacrylates, ethylene glycol dimethacrylate, vinyl esters of carboxylic acids, divinyl ether, pentaerythritol di-, tri-, or tetramethacrylate or acrylate, trimethylopropane trimethacrylate or acrylate, alkylene bis acrylamides or methacrylamides, methacrylic and acrylic acid, acrylamide, hydroxyethyl methacrylate and their mixtures. The polymerisation solvent is selected from aliphatic hydrocarbons, aromatic hydrocarbons, esters, alcohols, ketones, ethers, butyl alcohols, isobutyl alcohol, dimethyl sulphoxide, formamide, cyclohexanol, saccharose acetate isobutyrate, water, glycerol, sodium acetate, solutions of soluble polymers and mixtures thereof. The mixture is usually degassed for removal of oxygen and other dissolved gases by sonication or purging an inert gas such as nitrogen through the solution for a sufficient period of time. In a preferable method the polymerisation is performed using UV irradiation at temperature in the range of −30° C. to +60° C. for a period of from between about 2 minutes to 20 hours, depending on the intensity of irradiation. Furthermore, due to the living nature of dithiocarbamate ester-initiated polymerisation, a second and/or subsequent layers of addition polymer, copolymer or cross-linked polymer or copolymer can be additionally grafted over the initially grafted layer, optionally in the case of cross-linked polymer they may be prepared in the presence of one or more functional monomers and optionally in the presence of one or more template species, which may be ions, molecular species, complexes, macromolecules, peptides, proteins, crystals, spores, virus, yeast, bacterial or other template species known to those skilled in the art to form one or more molecularly imprinted polymer layers. In the case of surface-confined layers, grafted polymer may be applied to substantially change the surface characteristics of the layer, including modification of its hydrophobicity/hydrophilicity, its chemical functionality, catalytic properties, adhesive or non-adhesive nature, its solubility characteristics, its electrical conductivity, its magnetic or optical properties, its electrochromic, pH, temperature or solvent dependant behaviour or other “smart polymer” property or its ability to interact with other chemical, gaseous, ionic, inorganic or organic matter, including living matter, or some combination of these factors or any other such property known to those skilled in the art.

### Sixth Embodiment

The sixth embodiment of the present invention includes application of the described materials, articles, coatings, fabrics etc. The polymers can be prepared in insulating or conducting forms, depending on polymerization conditions. Depending on the material properties and application(s) required, the most important areas for utilisation of the materials of the present invention are (but are not limited to):
1. Surface-confined grafting on various surfaces e.g., metal electrodes, glass surfaces, polypropylene membranes, polystyrene surfaces etc.
2. Electroconductive coatings/connections for microelectronics and microarray sensors
3. Molecularly Imprinted polymers, Molecularly imprinted composite membranes
4. Soluble polyanilines, nanosized polyanilines
5. Soluble polyanilines for surface coating applications
6. Soluble polyaniline for sensor applications, including biosensor applications
7. Surface initiated dithiocarbamate mediated layer-by-layer controlled surface graft polymerisation
8. Flexible circuitry for electronics and personal electronics applications
9. Grafted polymers for various sensor applications, including but not limited to chemical sensors, gas sensors and affinity sensors, sensing materials in sensors and arrays i.e. gas sensors, electrochemical and optical sensors.
10. Initiators for making telechelic polymers, co-polymers and block-copolymers
11. Making hydride materials, composites
12. Catalysis and electrocatalysis
13. Materials for immobilisation and coated surfaces with high capacity for capturing target molecules
14. Selective and perm selective membranes, layers and coatings
15. Polymeric initiators and functional initiators
16. Materials for screen-printing
17. Electromagnetic protective shielding materials and coatings and materials and coatings, including fabrics and fabric coatings for dissipation of electrostatic charge
18. Novel sensing materials in sensors and arrays i.e. gas sensors and optical devices
19. Use in solid state batteries
20. Use as photorecording and photosensitive materials

### EXAMPLES

### Example 1

### Synthesis of N—(N′,N′-Diethyldithiocarbamoyl ethyl amido ethyl)-aniline (NDDEAEA), structure IIb

S-(carboxypropyl)-N-diethyl-dithiocarbamic acid was prepared in the following manner, adapted from the method disclosed by Hook et al. in U.S. Pat. No. 2,786,866: acrylic acid (0.50 mol, 36.00 g, 1 equiv), diethylamine (0.5 mol, 36.50 g, 1 equiv) and carbon disulfide (0.60 mol, 41.50 g, 1.1 equiv) in the exact order were added dropwise at 0° C. to solution of sodium hydroxide (0.50 mol, 20.00 g, 1 equiv) in 200 ml water. The mixture was stirred for thirty minutes at ambient temperature and then for 30 minutes at 60° C. (bath temperature). After cooling in ice bath, the solution is acidified with hydrochloric acid to pH 5.5. The oil formed solidified on vigorous stirring. This solid was filtered off and washed well with distilled water. The pale yellow crystals (30% yield) were dried. IR (KBr) cm⁻¹ (FT-IR spectra were obtained as KBr disks using a ThermoNicolet Avatar-370 spectrometer) 700-600 (C—S), 1500-1470 (N—C═S), 1420-1210 (COOH); ¹H NMR (NMR measurements are made using a Jeol ECX 400 MHz NMR) (400 MHz, CDCl3) δ 4.05-3.99 (2H, m, N(CH₂CH₃)₂), 3.75-3.73 (2H, m, N(CH₂CH₃)₂), 3.56 (2H, t, J=6.88, CH₂CH₂COOH), 2.87 (2H, t, J=5.96, CH₂CH₂COOH), 1.35-1.2 (6H, m, N(CH₂CH₃)₂) ppm; ¹³C NMR (400 MHz, CDCl₃) δ 195 (C(S)S), 178 (C(O)OH), 50 (NCH₂), 48 (NCH₂), 32 (SCH₂), 30 (CH₂C(O)OH), 13 (CH₂CH₃), 12 (CH₂CH₃) ppm; HRMS (provided by Medac Ltd, UK) (ES) Exact mass calculated C₁₅H₂₂N₂O₃S₂ [M+H]+: m/z: 221.05 (100.0%), 223.05 (9.1%), 222.06 (8.9%), 222.05 (2.0%). found: 221.05.

S-(carboxypropyl)-N-diethyl-dithiocarbamic acid, as prepared above, (0.004 mol, 0.922 g, 1 equiv) was dissolved into 20 ml of extra dry acetonitrile in an oven-dried 50 ml one-neck round-bottomed flask, equipped with magnetic stirring bar, argon atmosphere and covered with aluminum foil. To this mixture, N-phenylethylenediamine (0.004 mol, 0.545 g, 1 equiv) and 1-(3-dimethylaminopropyl)-3-ethyl-carbodiimide hydrochloride (0.008 mol, 1.6 g, 2 equiv) were added in this order. After three hours of stirring, the solvent was removed by evaporation. The crude mixture was dissolved in ethyl acetate and extracted five times with water, the organic layer dried with anhydrous sodium sulphate, filtered and the solvent evaporated in vacuo to obtain an oily product that solidifies to a waxy white material in 50% overall yield (98% purity). The product was used without any further purification. IR (KBr) (FT-IR spectra are obtained as KBr disks using a ThermoNicolet Avatar-370 spectrometer) 1647.26 (C═O), 1603.77 ((S)C—N), 1508.10 (N—C(O)), 1351.55 (C═S), 1268.93 (C—NC₆H₅), 981.92 (C—N), 755.80 ((S)C—S), 694.92 (S—CH₃) cm⁻¹; (NMR measurements were made using a Jeol ECX 400 MHz NMR) ¹H NMR (400 MHz, CDCl₃) δ 7.12 (2H, t, J=7.68 Hz, C₆H₅), 6.76 (1H, t, J=7.34 Hz, C₆H₅), 6.60 (2H, d, J=7.68 Hz, C₆H₅), 6.14 (1H, t, J=5.27, NH), 4.02-3.97 (2H, m, CH₂CH₃), 3.69-3.63 (2H, m, CH₂CH₃), 3.57 (2H, t, J=6.99, CH₂NHC₆H₅), 3.51 (2H, m, C(O)CH₂), 3.27 (2H, t, J=5.96, CH₂SC(S))_{,} 2.65 (2H, t, J=6.99, CH₂NHCO), 1.27-1.21 (6H, m, 2 CH₃) ppm; ¹³C NMR (100 MHz, CDCl₃) δ 195.2 (C(S)S), 171.8 (C(O)), 147.8 (C₆H₅), 129.3 (C₆H₅), 117.6 (C₆H₅), 112.8 (C₆H₅), 49.6 (CH₂NHC₅H₆), 46.8 (CH₂NHC(O)), 44.1 (CH₂CH₃), 39.0 (CH₂CH₃), 36.3 (CH₂SC(S)), 32.4 (CH₂C(O)), 12.3 (CH₃), 11.5 (CH₃) ppm; HRMS (ES) Exact mass calculated for C₂₆H₂₅N₃NaOS₂ [M+H]+: m/z: 362.13 (100.0%), 363.14 (17.6%), 364.13 (9.3%), 363.13 (2.7%), 364.14 (2.0%), 365.13 (1.7%). found: 362.1352 (100%), 140.1617 (42%), 363.1418 (40%) (Mass spectra obtained using a Waters LCT Premier XE mass spectrometer); elemental analysis (C₁₆H₂₅N₃OS₂): C, 56.60; H, 7.42; N, 12.38; O, 4.71; S, 18.89. found 1: C, 57.75; H, 7.60; N, 13.11; S, 18.39. found 2. C, 57.94; H, 7.69; N, 12.92; S, 18.11 (Elemental analysis was provided by Medac Ltd, UK.); R^{f}=0.24 (50% ethyl acetate/hexane).

### Example 2

### Preparation of S-methyl-N-methyl-N-(2-phenylamino)ethyl dithiocarbamate, (MMPAEDC), structure IIIa

N-methyl-N′-phenyl-1,2-ethylenediamine was first prepared by the method disclosed by Poindexter in U.S. Pat. No. 4,381,401, in the following manner: a mixture of aniline hydrochloride (13.00 g, 0.10 mol, 1 equiv), 3-methyl-2-oxazolidinone (11.00 g, 0.109 mole, 1.1 equiv) and 35 ml of 2-(2-methoxyethoxy)ethanol was placed in a 100 ml three-necked round-bottomed flask, equipped with magnetic stirring bar, condenser and thermometer. The mixture was heated to 170° C. in an oil bath. The reaction mixture became a yellow homogenous liquid and carbon dioxide evolution occurred on continued heating. After 17 hours, the reaction mixture was allowed to cool to room temperature. Most of the solvent was removed in vacuo to give a dark oily residue. This residue was taken up in 100 ml of a 10% aqueous sodium hydroxide solution and extracted with three portions of chloroform. The combined chloroform extracts were then dried over anhydrous sodium sulphate. After filtration, the chloroform was removed in vacuo to afford 20.4 g of the crude diamine as a brown liquid. Distillation at 105° C. to 110° C. (0.4 mm Hg) yielded the desired product as a clear liquid in 81% yield. ¹H NMR (400 MHz, MeOH-D₃), (NMR measurements were made using a Jeol ECX 400 MHz NMR) δ 7.84 (2H, t, J=8.37 Hz, C₆H₅), 7.34 (2H, d, J=7.68 Hz, C₆H₅), 7.29 (1H, t, J=6.53 Hz, C₆H₅), 3.86-3.82 (2H, q, J=6.08 Hz, CH₂NHC₆H₅), 3.43 (2H, t, J=6.30 Hz, CH₂NHCH₃), 3.08 (3H, s, CH₃), 2.37 (1H, s, NHCH₃) ppm; ¹³C NMR (100 MHz, MeOH-D₃) δ 146.23 (C₆H₅), 126.07 (C₆H₅), 112.73 (C₆H₅), 109.21 (C₆H₅), 47.67 (CH₂C₆H₅), 39.73 (CH₂NHCH₃), 33.20 (CH₃) ppm; DEPT 135 NMR (100 MHz, MeOH-D₃) δ 130.19 (up, C₆H₅), 116.85 (up, C₆H₅), 113.33 (up, C₆H₅), 51.79 (down, CH₂NHC₆H₅), 43.84 (down, CH₂NHCH₃), 37.32 (up, CH₃) ppm.

A mixture of sodium hydroxide (1.59 g, 39.9 mmol, 3 equiv) in 10 ml water, N-methyl-N′-phenyl-1,2-ethylenediamine (2.00 g, 0.0133 mol) and carbon disulfide (1.22 g, 15.98 mmol, 1.1 equiv) were placed in a 50 ml one-necked round-bottomed flask equipped with magnetic stirring bar under an argon atmosphere, at 0° C. After 30 minutes stirring, iodomethane (1.89 g, 13.3 mmol, 1 equiv), in 20 ml tetrahydrofuran was added at 0° C. After 30 minutes stirring, the mixture was allowed to warm to ambient temperature, and left to stir for 2 hours. The solvent was removed in vacuo. The mixture was dissolved in ethyl acetate and extracted three times with water. The ethyl acetate layer was collected, dried over anhydrous sodium sulphate, filtered and the solvent evaporated in vacuo. This procedure afforded the product, as a yellow oil (81% purity). Purification of the residue by column chromatography over silica-gel (25% ethyl acetate/hexane) afforded the product as a pale yellow oil, yield 55%. ¹H NMR (400 MHz, DMSO-D₆, 25° C.), (NMR measurements were made using a Jeol ECX 400 MHz NMR) δ 7.03 (2H, t, J=7.34 Hz, C₆H₅), 6.60-6.58 (2H, d, J=7.79 Hz, C₆H₅), 6.49 (1H, t, J=7.34 Hz, C₆H₅), 5.78 (0.33H, t, J=6.19 Hz, NHC₆H₅), 5.71 (0.67H, t, J=6.19 Hz, NH C₆H₅), 4.12 (1.35H, t, J=6.99 Hz, CH₂NHC₆H₅), 3.88 (0.65H, t, J=6.99 Hz, CH₂NHC₆H₅), 3.42 (1.07H, s, C(S)SCH₃) 3.34-3.23 (2H, m, CH₂N(CH₃)C(S)S), 3.29 (1.8H, s, C(S)SCH₃), 2.53 (3H, s, CH₃NC(S)S) ppm; ¹H NMR (400 MHz, DMSO-D₆, 130° C.) δ 6.96 (2H, s, C₆H₅), 6.52 (2H, s, C₆H₅), 6.41 (1H, s, C₆H₅), 5.18 (1H, s, NHC₆H₅), 3.99 (2H, s, CH₂NHC₆H₅), 3.29 (3H, s, C(S)SCH₃) 2.75 (2H, s, CH₂N(CH₃)C(S)S), 2.49 (3H, s, N(CH₃)C(S)S) ppm; ¹³C NMR (100 MHz, DMSO-D₃, 130° C.) δ 198.52 (C(S)S), 147.79 (C₆H₅), 128.28 (C₆H₅), 115.00 (C₆H₅), 111.89 (C₆H₅), 55.61 (CH₂NHC6H₅), 40.80 (CH₂N(CH₃)C(S)S), 39.63 (CH₃SC(S)) 19.81 (N(CH₃)C(S)S) ppm; DEPT 45 NMR (100 MHz, DMSO-D₃) δ 129.52 (C₆H₅), 116.89 (C₆H₅), 112.49 (C₆H₅), 56.23 (CH₂NHC₆H₅), 41.21 (CH₂N(CH₃)C(S)S), 39.63 (CH₃SC(S)), 20.39 (N(CH₃)C(S)S) ppm; DEPT 90 NMR (100 MHz, DMSO-D₃) δ 129.52 (C₆H₅), 116.39 (C₆H₅), 112.49 (C₆H₅) ppm; DEPT 135 NMR (100 MHz, DMSO-D₃) δ 129.52 (up, C₆H₅), 116.39 (up, C₆H₅), 112.49 (up, C₆H₅), 56.23 (down, CH₂NHC₆H₅), 41.21 (down, CH₂N(CH₃)C(S)S), 39.63 (up, CH₃SC(S)), 20.39 (up, N(CH₃)C(S)S) ppm. HRMS (ES) Exact mass calculated for C₁₁H₁₆N₂S₂ [M+H]+: m/z: 240.08 (100.0%), 241.08 (12.1%), 242.07 (9.1%), 241.07 (2.3%), 243.07 (1.2%). found: 241.08 (60%), 210.15 (100%) (Mass spectra was provided by Medac Ltd, UK); elemental analysis (C₁₁H₁₆N₂S₂): C, 54.96; H, 6.71; N, 11.65; S, 26.68. found 1: C, 54.07; H, 7.51; N, 11.63; S, 24.50. found 2: C, 53.88; H, 7.68; N, 11.60; S, 24.66 (Elemental Analysis was provided by Medac Ltd, UK.); R_{f}=0.3 (25% ethyl acetate/hexane).

### Example 3

### Electropolymerisation of the Aniline Group of NDDEAEA

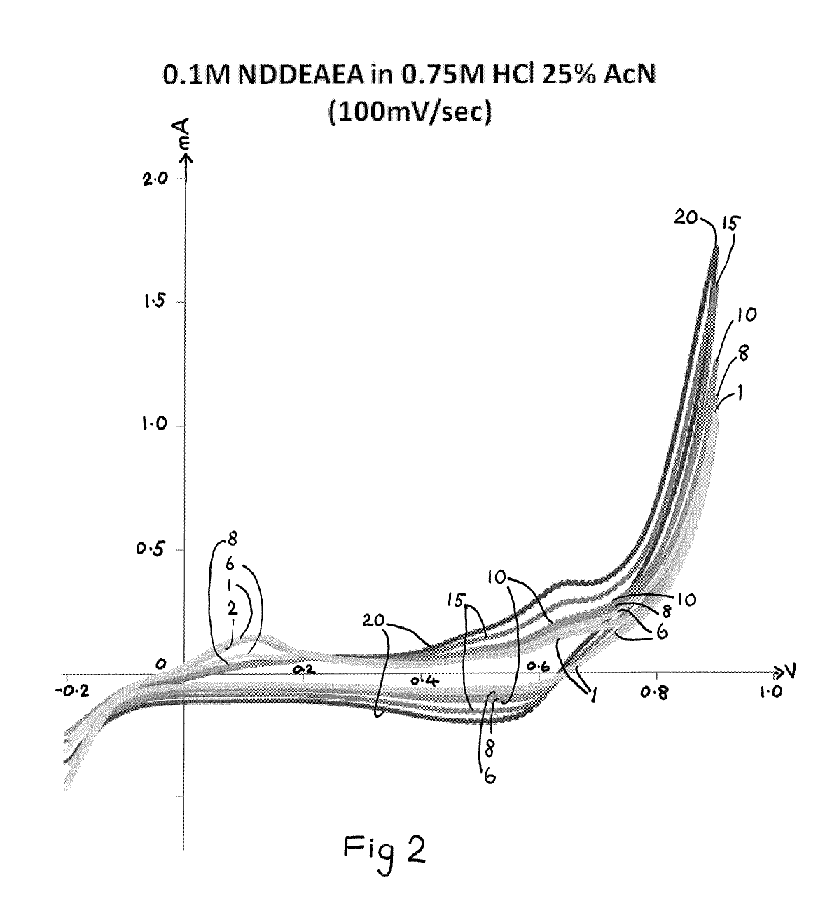

An Autolab PSTAT-10 instrument (Eco-Chemie BV, Utrecht, Netherlands) was utilised for all electrochemical experiments. A conventional screen printed gold electrode (SPE) (1.6 mm diameter, from Dropsens) was used, which comprised a gold working electrode and silver counter and reference electrodes. Before polymerisation each new electrode was cleaned and pre-treated by potential cyclic by CV from 0 to +0.7 V, 5 times cycling, 50 mV/S scan rate in 1.5 M HCl. Electrodes were cycled (20 times) between −0.2 V and +0.9 V at a scan rate of 100 mV/s, (step potential 7 mV) in 0.2 M 25% acetonitrile solution of NDDEAEA in 0.56M HCl. Solutions were mixed well and degassed for 10 minutes by purging with Ar or Nitrogen before experiments and covered with aluminium foil to protect from light. CV measurements were performed by placing 150 μL of the test solution of NDDEAEA onto the surface of SPE for electropolymerisation. Electropolymerisation was carried out under Ar atmosphere and in the dark. Electropolymerised films were rinsed with deionised water (once), dried in a stream of nitrogen and stored dry in dark. The electrode bearing the electropolymerised polyaniline films with diethyldithiocarbamate ester groups was washed with water. FIG. 2 shows the CV obtained during the electropolymerization (20 cycles) of NDDEAEA in a 3:1 mixture of 0.75M HCl and acetonitrile solution, clearly displaying oxidation and reduction peaks at +0.65 V and +0.52 V (vs Ag/AgCl) respectively.

### Example 4

### Surface-Confined Photo Grafting of Various Addition Polymers on Electropolymerised Films of NDDEAEA

An electropolymerised poly(NDDEAEA)-modified SPE was placed horizontally in a glass vial and treated under dark condition with 150 μL of 0.1 M unsaturated monomer in acetonitrile. Monomers used were methacrylic acid (MAA), 2-acrylamido-2-methylpropane sulphonic acid (AMPSA) and styrene. The monomer solutions purged with nitrogen for 10 minutes to remove oxygen before irradiation. The vial was sealed with Parafilm® and purged with nitrogen to maintain an inert atmosphere over the solution during irradiation. The electrode surface was then UV-irradiated for 20 minutes with a fiber optic light source (300W CERMAX xenon arc lamp). The photografted electrodes were then rinsed in a mixture of 50:50 v/v methanol:deionised water and dried in a stream of nitrogen. Contact angle measurements were used to characterise changes that had occurred in the functionality and hydrophobic/hydrophilic nature of the polymeric film after grafting.

a) Photografting of poly(methacrylic acid) to an electropolymerised film of NDDEAEA: An electropolymerised film of NDDEAEA was treated with a solution of methacrylic acid (150 μL of 0.1 M in acetonitrile) and grafted as described above. The poly(MAA) grafted layer was moderately hydrophilic with a contact angle of 45° (Table 1).

b) Photografting of poly(AMPSA) to an electropolymerised film of NDDEAEA: An electropolymerised film of NDDEAEA was treated with a solution of AMPSA (150 μL of 0.1 M in acetonitrile) and grafted as described above. The poly(AMPSA) grafted layer was very hydrophilic with a contact angle of 33° (Table 1).

c) Photografting of poly(Styrene) to an electropolymerised film of NDDEAEA: An electropolymerised film of NDDEAEA was treated with a solution of styrene (150 μL of 0.1 M in acetonitrile) and grafted as described above. The poly(Styrene) grafted layer was hydrophobic with a contact angle of 64.3° (Table 1).

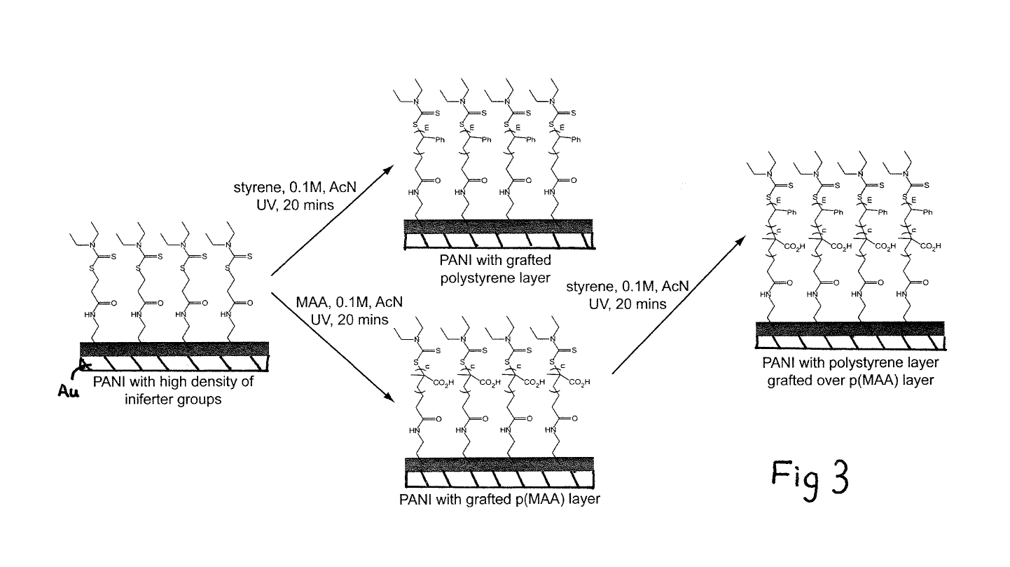

d) Layer-by-layer consecutive photografting of poly(styrene) over poly(MAA) layer grafted to an electropolymerized film of poly(NDDEAEA). To prove the living nature of the dithiocarbamate ester groups of poly(NDDEAEA), activated during UV irradiation, consecutive layer-by-layer grafting of solutions of two different monomers, viz., methacrylic acid followed by styrene, were performed. For the layer-by-layer grafting experiment, a poly(MAA) grafted SPE electrode, prepared as described in part a) above, was treated with a solution of styrene (150 μL of 0.1 M in acetonitrile) as described above in part c) (FIG. 3). The contact angle of the layer-by-layer grafted film was consistent with the second layer, being measured as 64.5°.

TABLE 1
Advancing water contact angles in air for bare
gold electrodes and various surface-grafted polymers.

| | Water contact angle |
|---|---|
| Nature of the surface | (°) |
| Pretreated Screen-printed gold Electrode | 66.0 |
| (SPE) | |
| Electropolymerised poly(NDDEAEA) | 29.4 |
| Poly(MAA) surface-grafted to | 45.1 |
| electropolymerised poly(NDDEAEA) film | |
| Poly(AMPSA) surface-grafted to | 33.0 |
| electropolymerised poly(NDDEAEA) film | |
| Poly(Styrene) surface-grafted to | 64.3 |
| electropolymerised poly(NDDEAEA) film | |
| Poly(Styrene) over poly(MAA) surface- | 64.5 |
| grafted to electropolymerised | |
| poly(NDDEAEA) film (layer-by-layer | |
| grafting) | |

### Example 5

### Polymerization of NDDEAEA Using Chemical Oxidation for the Formation of Thin Transparent Coatings on Various Supports Such as Polystyrene Micro-Titre Plates, Cuvettes and Poly(Propylene) Filtration Membranes

a) Effect of Conditions on Chemical Oxidative Polymerisation of NDDEAEA:

Chemical oxidative polymerization was performed under various conditions as follows:

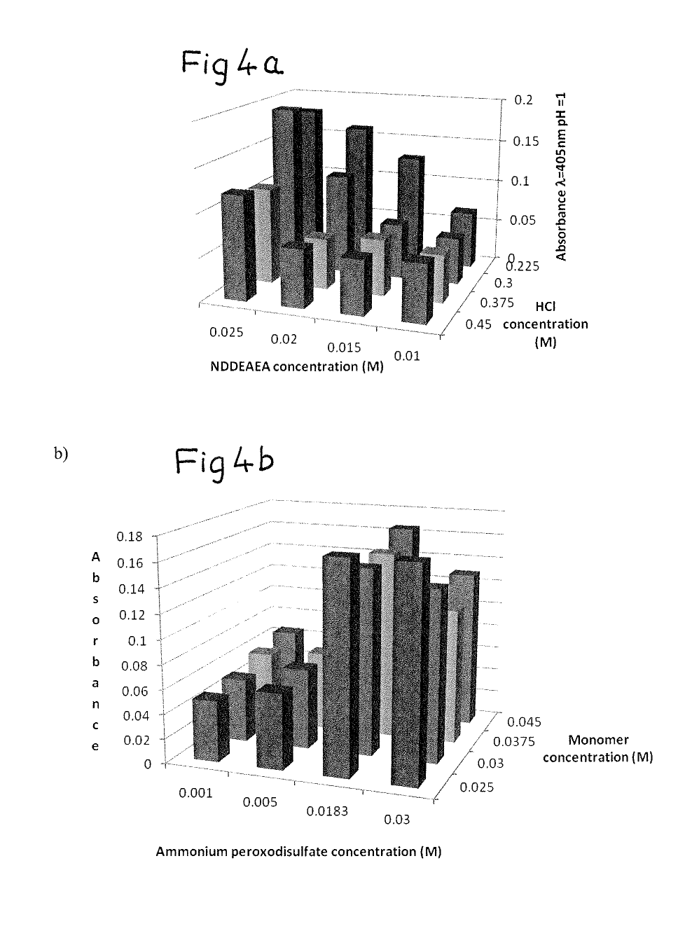

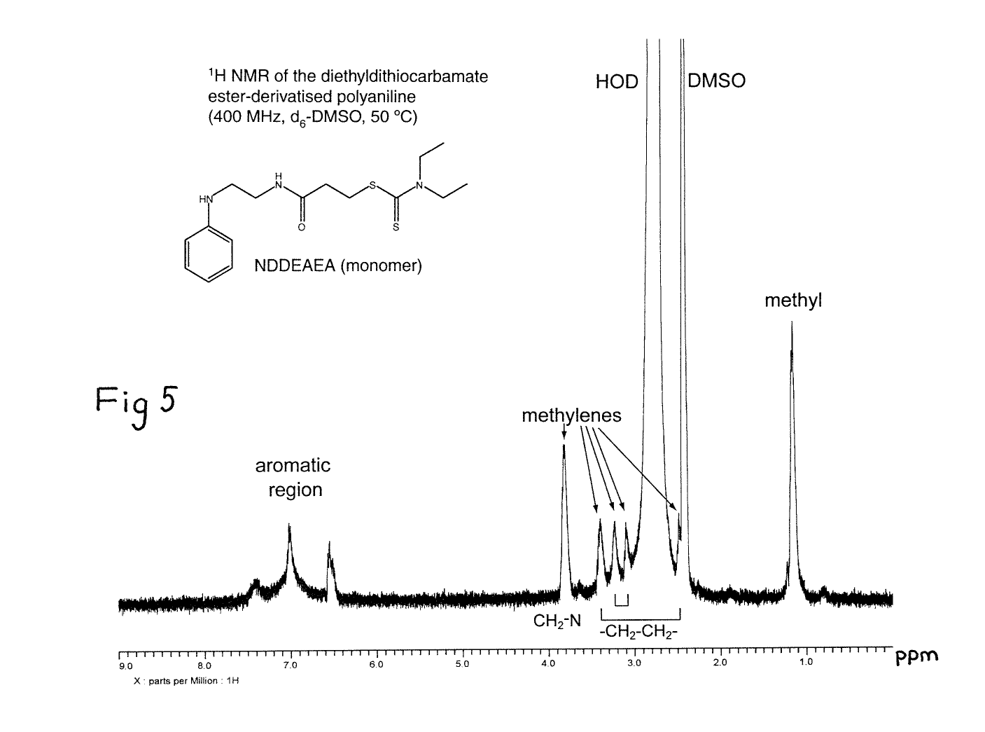

The effect of monomer (NDDEAEA) concentration and HCl concentration on the polymerisation of NDDEAEA by oxidation with ammonium persulphate (0.0183 M) was assessed by preparing solutions in which the concentration of NDDEAEA was varied between 0.01 M and 0.025 M and the concentration of HCl was varied between 0.225 M and 0.45 M in a 4×4 grid. Polymerisation was conducted in 25% acetonitrile in water for 2 h in the dark at room temperature. Bluish-green films of poly(NDDEAEA) were seen to form on the inner surfaces of the cuvettes or micro-titre plate wells or on the surface of polypropylene ultrafiltration membranes. Poly(NDDEAEA) films were washed with water and 1 M HCl. Optical densities were recorded using a microplate reader (for micro-titre plates) or by UV spectrophotometry (UV-1800 Shimadzu) (for cuvettes) under various pH conditions. Similarly the effect of varying the ammonium persulphate concentration between 0.001 M to 0.03 M and NDDEAEA concentration, varied between 0.025 M to 0.045 M, was assessed at a concentration of 0.225 M, also in a 4×4 grid. The optical densities at 405 nm and pH=1 of chemically polymerised poly(NDDEAEA) films deposited in micro-titre plate wells is shown in FIGS. 4a and b. Contact angle decreased with decreasing pH. In the present study, poly(NDDEEA)-coated micro-titre plate wells at basic pH (12.0) were relatively hydrophobic with a contact angle of 82.3°, whereas at pH 1.0 it was 39.52 °.

### Example 6

### Photografting of a Molecularly Imprinted Polymer (MIP) Over Chemically Polymerised Poly(NDDEAEA) Deposited on a Polypropylene Membrane

Polypropylene (PP) ultrafiltration membranes (with a nominal cut-off pore diameter of 0.2 μm and thickness of 150 μm, PP 2E-HF, Membrana, Germany) were cut into small discs of 47 and 25 mm diameter. Membranes were pre-treated with methanol for 1 h in a petri dish. Oxidative polymerisation was performed using ammonium peroxodisulfate (APS) under the following conditions: 0.0183 M APS, 0.025 M NDDEAEA, 0.225 M HCl, 25% acetonitrile in water, 1.45 h polymerisation in the dark at room temperature. This resulted in the deposition of a thin, bluish-greenish layer of functionalised polyaniline coating the membrane surfaces. Poly(NDDEAEA)-coated PP membranes were washed with water and 0.1 M HCl three times. The degree of grafting was determined gravimetrically to characterise the amount of grafted polymers on the membrane surface. The following equation was used:

DG(%)=(W₁−W₀)/W₀×100%

where W₀ and W₁ represent the weights of the membrane before and after grafting, respectively.

DG of functionalised polyaniline-grafted PP membranes was 13.28%.

Photografting polymerisation of MIP was performed on poly(NDDEAEA)-grafted PP membranes in acetonitrile. Time of UV irradiation was optimised as 45 mins.

The pre-weighed Poly(NDDEAEA)-coated membranes were immersed in a petri dish containing 3 ml monomer solution (deaereated by bubbling nitrogen for 10 mins) and the petri dish was placed inside a box supplied with an inlet for continuous flow of nitrogen throughout the period of polymerisation and the system was UV irradiated (Philips UV lamp) for 45 mins. The polymerisation mixture contained atrazine (10 mg) as a template, 20 mg methacrylic acid (as functional monomer), and 380 mg ethylene glycol dimethacrylate (as cross-linker) in acetonitrile. Similarly, a reference polymer (blank) membrane was prepared as above but in the absence of atrazine. Template was removed by overnight soxhlet extraction in methanol and washing the grafted membranes in 0.1 M acetic acid in methanol solution for 4 h on a shaker. LC-MS and HPLC was used to determine whether the template was still leaching from the membrane. After drying at 45° C. overnight, DG was calculated as 6.67% (2 mg increase for a 47 mm diameter membrane, polymer grafting density 1.15 μg mm⁻²). Water contact angle were measured before and after grafting of MIP over the PP membranes (Table 2).

MIP membrane recognition properties were quantified by HPLC and evaluated by measuring their capability to adsorb herbicides from aqueous solution during filtration. MIP membranes showed 94% binding for 125 μg/mL atrazine while non-imprinted (blank) reference membranes bound only 15% under the same conditions.

TABLE 2
Advancing water contact angles in air for
polypropylene membranes grafted with poly(NDDEAEA) and
poly(NDDEAEA) grafted with an atrazine-imprinted polymer (MIP) film.

| Membrane surface chemistry | Water contact angle (°) |
|---|---|
| Unmodified PP membrane | 138.5 |
| Poly(NDDEAEA) grafted layer | 69.4 |
| Atrazine-MIP (MAA-EGDMA) | 57.6 |
| photografted over | |
| poly(NDDEAEA) | |

### Example 7

### Preparation of a Soluble Polyaniline Following Chemical Polymerisation of NDDEAEA

In a typical procedure, 0.75 mL of 0.1M solution of NDDEAEA in acetonitrile was dissolved in 0.68 mL of 1M HCl in a glass vial. A solution comprising 0.98 mL of water and 0.6 mL of (0.0915 M) ammonium peroxodisulfate was added dropwise to the acidified solution of NDDEAEA, over a period of 10 min, with constant stirring. The mixture was agitated on a vortex for the first 1 h and then left on a shaker with gentle agitation for a further 1 h during which time polymerisation took place. The reaction product was separated by centrifugation and dried. It was completely soluble in methanol, DMF, DMSO, THF, chloroform, dichloromethane and partially soluble in acetonitrile and water. The size of the polymer particles were measured using a nanosizer instrument (Malvern) and found to be around 100 nm. The effects of pH and redox sensitivity were checked by the addition of 100 μL of HCl and NaOH solution to the soluble poly(NDDEAEA) in methanol (pH 1.0, 7.0 and 10.0) and colour of the solution was changed from green to blue when the pH was increased from 1.0 to 10.0. The molecular weight of the polymer was estimated by GPC (with respect to polystyrene standards) and was shown to be >100,000.

The ¹H NMR (in d₆-DMSO) and the UV spectrum (in methanol) of the soluble poly(NDDEAEA) were recorded, which confirmed the presence of the diethyldithiocarbamate ester pendant groups in the poly(NDDEAEA) structure following chemical polymerisation.

The invention has been described with reference to preferred embodiments. The skilled reader will appreciate that various modifications and alternatives are possible within the scope of the invention, and it is intended to cover all such modifications and alternatives by the appended claims.

1) A monomer having an aniline moiety linked through the aniline nitrogen to a dithiocarbamate moiety, or a salt thereof wherein the aniline nitrogen is protonated.

2) A monomer or salt according to claim 1 wherein the aniline moiety is linked to a sulfur atom of the dithiocarbamate via a spacer.

3) A monomer or salts according to claim 2 wherein the monomer is of formula (A):
Ar—NH-[SPACER]-S—C(═S)—NR¹R² (A)
where [SPACER] represents a divalent group comprising a chain of 2-7 atoms; Ar is a phenyl group which is unsubstituted or has up to 4 substituents; and the groups R¹ and R² are independently selected from groups —CH₂—R⁵ where R⁵ is selected from hydrogen and optionally substituted straight or branched alkyl groups; or R¹ and R² are linked to form a cyclic structure.

4) A monomer or salt according to claim 3 wherein the spacer comprises a chain of 2-7 carbon atoms, one or more of which may be replaced by a heteroatom, wherein some of the atoms in the chain may be functionalised or bear substituents.

5) A monomer or salt according to claim 2 wherein the spacer comprises an ester or amide linkage.

6) A monomer or salt according to claim 2 wherein the spacer is selected from
—CH₂—CH₂—X—CO—CH₂—CH₂—
(in either orientation), where X is O or NH;
—(CH₂)ₙ—
where n is 2-7;
—(CH₂)ₐ—O—(CH₂)_{b}—
where a and b are integers of 1-5 and a+b is 2-6; and variants in which one or more methylene groups are substituted.

7) A monomer or salt according to claim 1 wherein the aniline moiety is linked to the nitrogen atom of the dithiocarbamate via a spacer.

8) A monomer or salt according to claim 7 wherein the monomer is of formula (B):
Ar—NH-[SPACER]-NR³—C(═S)—S—R⁴
where [SPACER] is a divalent group selected from methylene, substituted methylene, and a group comprising a chain of 2-7 atoms; Ar is a phenyl group which is unsubstituted or has up to 4 substituents; R³ is a group of the form —CH₂R⁵ where R⁵ is selected from hydrogen and optionally substituted straight or branched alkyl groups; and R⁴ is an optionally substituted straight or branched alkyl group.

9) A polyaniline produced by oxidative polymerisation of one or more monomers having an aniline moiety linked through the aniline nitrogen to a dithiocarbamate moiety, or a slat thereof, optionally together with one or more comonomers.

10) A polyaniline according to claim 9 which has a conjugated polyaniline backbone and bears a multiplicity of dithiocarbamate moieties.

11) A polyaniline according to claim 9 wherein a said monomer is of formula (A):
Ar—NH-[SPACER]-S—C(═S)—NR¹R² (A)
where [SPACER] represents a divalent group comprising a chain of 2-7 atoms; Ar is a phenyl group which is unsubstituted or has up to 4 substituents; and the groups R¹ and R² are independently selected from groups —CH₂—R⁵ where R⁵ is selected from hydrogen and optionally substituted straight or branched alkyl groups; or R¹ and R² are linked to form a cyclic structure.

12) A polyaniline according to claim 9 wherein a said monomer is of formula (B):
Ar—NH-[SPACER]-NR³—C(═S)—S—R⁴
where [SPACER] is a divalent group selected from methylene, substituted methylene, and a group comprising a chain of 2-7 atoms; Ar is a phenyl group which is unsubstituted or has up to 4 substituents; R³ is a group of the form —CH₂R⁵ where R⁵ is selected from hydrogen and optionally substituted straight or branched alkyl groups; and R⁴ is an optionally substituted straight or branched alkyl group.

13) A method of producing a polyaniline which has a conjugated polyaniline backbone and bears a multiplicity of dithiocarbamate moieties comprising oxidative polymerisation of one or more monomers having an aniline moiety linked through the aniline nitrogen to a dithiocarbamate moiety, or a salt thereof, optionally together with one or more comonomers.

14) A method according to claim 13 which employs electropolymerisation to deposit a layer of polyaniline on an electrode surface.

15) A method according to claim 13 which employs a chemical oxidant, and conditions are selected so that the polyaniline is produced in the form of a film, powder, particles, microparticles, nanoparticles, microcapsules, microtubes, microrods, nanotubes, nanorods or as a soluble polymer.

16) A method according to claim 13 including a subsequent step of using the polyaniline as an iniferter and grafting one or more addition polymers onto it.

17) A method according to claim 16 wherein a said addition polymer is a molecularly imprinted polymer.
